(19) United States
(12) Patent Application Publication
(10) Pub. No.: US 2021/0353795 A1
(43) Pub. Date: Nov. 18, 2021

(54) CONTAINER WITH AUTOMATIC STERILIZATION FUNCTION

(71) Applicant: Po-Chun Huang, Taipei City (TW)

(72) Inventor: Po-Chun Huang, Taipei City (TW)

(21) Appl. No.: 16/877,469

(22) Filed: May 18, 2020

(51) Int. Cl. A61L 2/10; A61L 2/24

(52) U.S. Cl. CPC A61L 2/10; A61L 2/24; A61L 2202/16; A61L 2202/14; A61L 2202/11

(57) ABSTRACT

The present invention provides a container with automatic sterilization function, the container is combined with a first cover body and a second cover body which are connected to each other. The first cover body and the second cover body can be lifted up by using a hinge structure, and combined by using a set of catching groove and snap. The second cover body and container are combined tightly or separated by screwing opposite screw thread sections. The inner face of inner chamber of the first cover body has a reflective layer made of reflecting material. A circuit structure is disposed in the main body of the second cover body (and in the inner chamber of the first cover body). A suction port is disposed on the surface of the second cover body towards the first cover body, more than one first UV light source structure is disposed around to irradiate the suction port. Another surface towards the container is provided with a second UV light source structure irradiating the inside of container. All of the first UV light source structures and the second UV light source structures are linked with the circuit structure to provide timed sterilization function. In addition, the second cover body is provided with a first sensor for detecting the open/close of the first cover body as the basis of on/off of the first UV light source structure; and a second sensor for detecting the separation/combination of the second cover body and container as the basis of on/off of the second UV light source structure.

### BACKGROUND OF INVENTION

### 1. Field of the Invention

The present invention relates to a safer and more practical container, and more particularly to a container with automatic sterilization function.

### 2. Description of Related Art

The articles used in daily life often have bacteria or microbes that cannot be detected with naked eye, which may cause infections or diseases without sound sterilization. Based on rising awareness of personal hygiene and safety, more and more people usually look for periodical sterilization of articles which are contacted or used constantly even environment, especially the articles contacted and used by children.

At present, some manufacturers have developed products using the bactericidal power of UV light, the UV light is embedded in the cover body of a bottle (e.g. thermos bottle), it is initiated by pressing for sterilization. However, the sterilization function of this type of commercially available products is too simple and incomplete, the following deficiencies can be found by looking at the structure carefully, the UV light can sterilize the water loaded, but the user's mouth contacts the thermos bottle mouth, leaving a lot of bacteria, and the whole bottle mouth is contaminated after the user drinks a few times, even if the water is sterilized in advance, when the user is drinking, the water flows across the bottle mouth into the mouth, bringing bacteria into human body. When the person has worse immunity or condition, he is likely to get sick. Secondly, the UV light is embedded in the cover body of bottle, it is actuated to emit light by pressing the switch, but when the cover body is separate from the bottle body, the press switch is still manipulatable. Thus, when the cover body and bottle body are being separated or assembled, if the switch is touched by accident, the UV light will emit light, harming human skin and eyes, especially the thermos bottles or water bottles for children, if the luminescence mechanism cannot be controlled safely, the children may play the UV emission or intermittent luminescence as a game, pressing the switching continuously, shortening the service life of the article, even injuring the eyes and causing blindness. The UV can be used as a tool to kill bacteria and microbes, but it is harmful to human body to some extent, it must be used carefully.

### SUMMARY OF THE INVENTION

The first object of the present invention is to provide a container with automatic sterilization function, which positively and fully sterilizes the constant contact sites of the container, so as to obtain safer protection.

The second object of the present invention is to provide a container with automatic sterilization function, the UV light source structure is in timed luminescent sterilization state before the container and cover body are opened or separated, once the cover body is opened, it is turned off immediately. When the original state is restored, the timed sterilization is actuated, so as to avoid being played by children, and to completely eliminate the probability of accidental injury to human body induced by UV exposure.

The third object of the present invention is to provide a container with automatic sterilization function, which can be provided with a straw for children's convenience.

The fourth object of the present invention is to provide a container with automatic sterilization function, which can be applied to containers of different types and sizes, including bottles, jars, and cups, so as to be used by common people with different requirements safely.

To attain said objects, this inventor designed a safe and practical container with automatic sterilization function, including the following two specific means. In the first embodiment, the opening of the container is combined with a first cover body and a second cover body which are connected to each other. The inner face of inner chamber of the first cover body has a reflective layer made of an arbitrary reflecting material. A circuit structure is disposed in the main body of the second cover body. A suction port is disposed on the surface of the second cover body towards the first cover body, at least more than one first UV light source structure irradiating the inner chamber of the first cover body and at least more than one first UV light source structure irradiating the circumference of the suction port are arranged by the suction port, another surface of the second cover body towards the container is provided with more than one second UV light source structure.

In the second embodiment, the opening of the container is combined with a first cover body and a second cover body which are connected to each other. A first UV light source structure is disposed on the inner face of inner chamber of the first cover body. A circuit structure is disposed in the main body of the second cover body, and a suction port is disposed on the surface of the second cover body towards the first cover body. At least more than one first UV light source structure irradiating the circumference of the suction port is arranged by the suction port. More than one second UV light source structure is disposed on another surface of the second cover body towards the container.

In the above two embodiments, a coupling section and a catching groove opposite to each other are arranged around the first cover body. A fixing component and a snap are arranged oppositely around the second cover body. The fixing component and the coupling section form a hinge. The snap can be combined with the catching groove, so that the first cover body can be opened and closed. The inner face of the second cover body for combining the container has a screw thread section. The opening circumference of the container has a corresponding screw thread section, so that the second cover body can be coupled tightly or separated.

In the above two embodiments, a first sensor is disposed in the snap for combining the first cover body with the second cover body, the on/off of the first UV light source structure is subject to the open/close of the first cover body. In the combination of the second cover body and container, a screw thread section corresponding to the container opening is disposed on the inner face of the second cover body, and a second sensor is disposed at the edge of the end edge of the screw thread section connected to the main body, the on/off of the second UV light source structure is subject to the separation/combination of the second cover body and container.

In said two embodiments, the circuit structure is disposed in the main body of the second cover body (or in the inner chamber of the first cover body according to requirement or practice). The circuit structure at least comprises a controller (chip), a timer, a relay, a charge power supply and a Bluetooth module. All of the first UV light source structure, the second UV light source structure, the first sensor and the second sensor are electrically connected to the circuit structure, so as to obtain the required power source and to perform various functions positively.

In said two embodiments, the sterilization time of the first UV light source structure can be set up and adjusted freely, usually within 1 minute, the optimal range is 10˜20 seconds. The sterilization time of the second UV light source structure is usually within 3 minutes, and the optimal range is 90˜120 seconds. In implementation, the Bluetooth module is assorted with the mobile device, the sterilization time is adjusted freely according to the sterilizing effect for the containers of different capacities by operating the built-in APP.

In said two embodiments, the quantity and the orientation and tilt angle of the first UV light source structure irradiating the circumference of suction port on the second cover body are unrestricted, so as to irradiate the overall circumference of the suction port completely to implement positively perfect sterilizing effect.

In said two embodiments, an upright ring surface is formed around the second cover body, the more than one first UV light source structure irradiating the circumference of suction port is embedded in the upright ring surface.

In said two embodiments, the suction port can be provided with a straw.

In said two embodiments, the container can be any one of bottle, jar, cup and barrel of different capacities.

In the use of the container with automatic sterilization function, the first cover body and the second cover body, the second cover body and the container are a closed sterilizing space respectively. Between the first cover body and the second cover body, the suction port and periphery are irradiated by multiple first UV light source structures simultaneously, so as to implement favorable timed sterilizing effect on the inside and outside. Between the second cover body and container, the liquid loaded in the container is irradiated by at least more than one second UV light source structure above, so as to implement the required favorable timed sterilizing effect. In the present invention, as the suction port is the unique part the user's mouth contacts, the sterilizing effect on the suction port will directly influence the user's physical health status. The all-directional UV light disposition design has the best sterilizing effect on the suction port contacted repeatedly. Moreover, the secondary sterilization design for the liquid loaded in the container makes drinking safer for the user.

### BRIEF DESCRIPTION OF THE DRAWINGS

FIG. 1 is the exploded diagram of the appearance of the container with automatic sterilization function in a preferred embodiment.

FIG. 2 is the schematic diagram of disposition of the second cover body and the first UV light source structure in a preferred embodiment.

FIG. 3 is the schematic section of the container, the first cover body and the second cover body in a preferred embodiment.

FIG. 4 (A) is the schematic diagram (1) of service mode of the container in a preferred embodiment.

FIG. 4 (B) is the schematic diagram (2) of service mode of the container in a preferred embodiment.

FIG. 4 (C) is the schematic diagram (3) of service mode of the container in a preferred embodiment.

FIG. 5 is the schematic diagram of another implementation pattern of the first UV light source structure on the second cover body in a preferred embodiment.

FIG. 6 is the schematic diagram of another implementation pattern of the first UV light source structure on the second cover body in a preferred embodiment.

FIG. 7 is the exploded diagram of the appearance of the container with automatic sterilization function in another preferred embodiment.

FIG. 8 is the schematic diagram of the second cover body and the first UV light source structure in another preferred embodiment.

FIG. 9 is the schematic section of the container, the first cover body and the second cover body in another preferred embodiment.

FIG. 10 is the schematic diagram of another implementation pattern of the first UV light source structure on the second cover body in another preferred embodiment.

FIG. 11 is the schematic diagram of another implementation pattern of the first UV light source structure on the second cover body in another preferred embodiment.

### DETAILED DESCRIPTION OF THE INVENTION

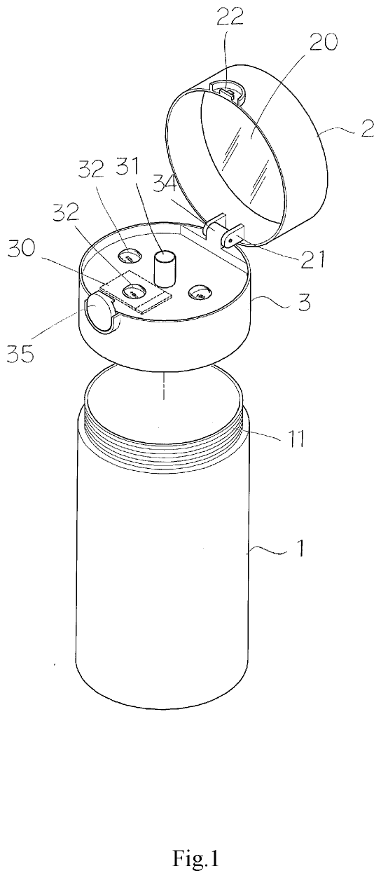

The first preferred embodiment in FIG. 1 presents the external structure of the container with automatic sterilization function of the present invention. In the present invention, the container (1), taking a bottle as an example, such as a thermos bottle, but not limited to this, in other embodiments, the container (1) can be a jar, a cup, a barrel or a pot, and various types have arbitrary capacities and sizes.

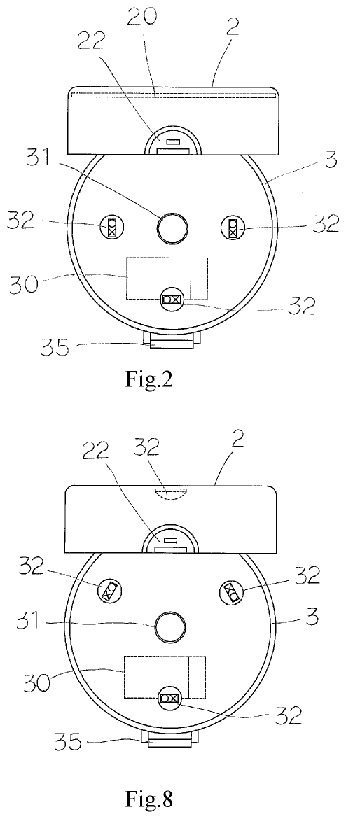

As shown in FIG. 1 and FIG. 2, the outer peripheral surface of opening of the container (1) is provided with a screw thread section (11), combined with a first cover body (2) and a second cover body (3) which are connected to each other. The inner face of inner chamber of the first cover body (2) has a reflective layer (20) made of an arbitrary reflecting material. A circuit structure (30) is disposed in the main body of the second cover body (3), and an upright suction port (31) is disposed on the surface of the second cover body (3) towards the first cover body (2). In this embodiment, the suction port (31) can be made of appropriate materials for different users, such as adults or children, so as to provide better touch. At least more than one first UV light source structure (32) irradiating the reflective layer (20) in the first cover body (2) and at least more than one first UV light source structure (32) irradiating the circumference of the suction port (31) are arranged around the suction port (31), another surface of the second cover body (3) towards container (1) is provided with more than one second UV light source structure (33) (not shown in FIG. 1).

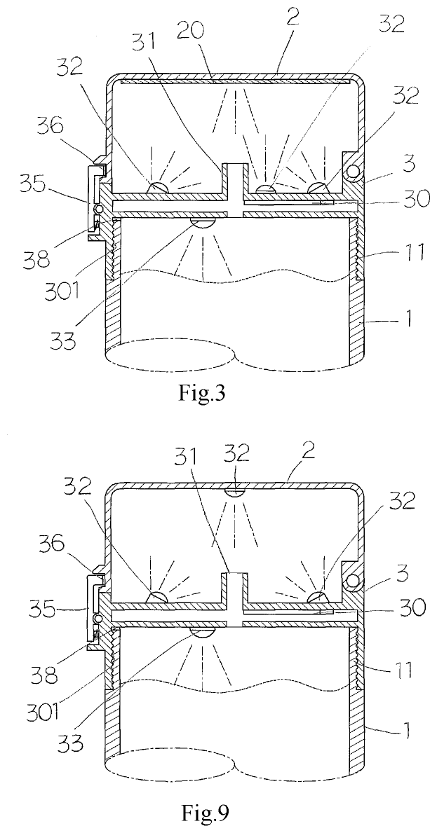

As shown in FIG. 1 and FIG. 3, a coupling section (21) and a catching groove (22) opposite to each other are disposed around the first cover body (2), and a fixing component (34) and a snap (35) are designed oppositely around the second cover body (3). The fixing component (34) and the coupling section (21) form a hinge, so that the first cover body (2) can be lifted or closed. The snap (35) can be combined with the catching groove (22), so that the first cover body (2) can be fastened to or disconnected from the second cover body (3). A first sensor (36) is laid in the snap (35) for sensing the opening or closing of the first cover body (2) as the basis of on/off of all the first UV light source structures (32). The inner face of the second cover body (3) corresponding to the opening of container (1) has a screw thread section (301), and a second sensor (38) is disposed on the edge of the end of the screw thread section (301) connected to main body. The opening circumference of the container (1) has a corresponding screw thread section (11) for combining or loosening the screw thread section (301) of the second cover body (3). The second sensor (38) detects the separation or combination of the second cover body (3) and container (1) as the basis of on/off of the second UV light source structure (33).

Said circuit structure (30) is embedded in the main body of the second cover body (3) in this embodiment, at least comprising a controller (chip), a timer, a relay, a charge power supply and a Bluetooth module. All of the first UV light source structure (32), the second UV light source structure (33) and the first sensor (36) and the second sensor (38) are electrically connected to the circuit structure (30), so as to obtain the required power source and to execute function. In addition, the first cover body (2) and the second cover body (3) are all provided with water proofing, so as to protect the circuit structure (30) against damp and damage.

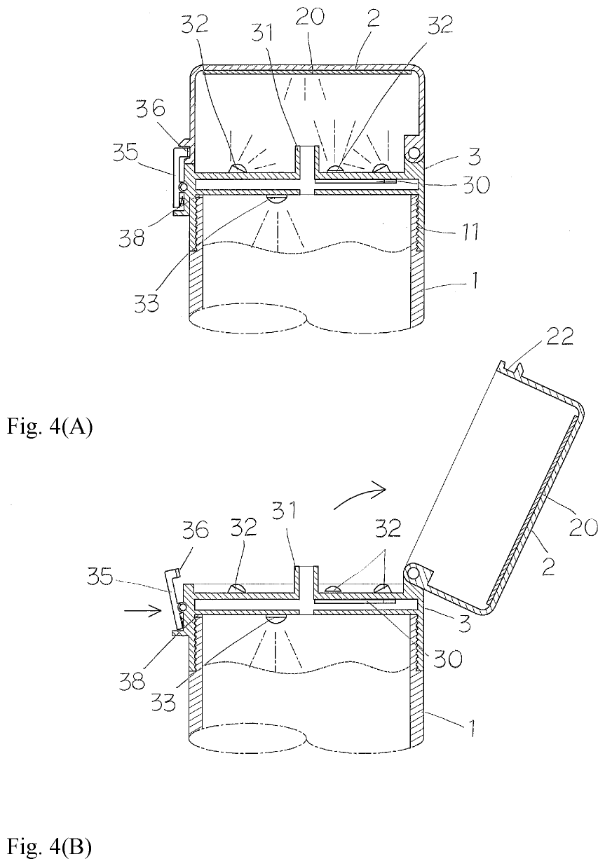

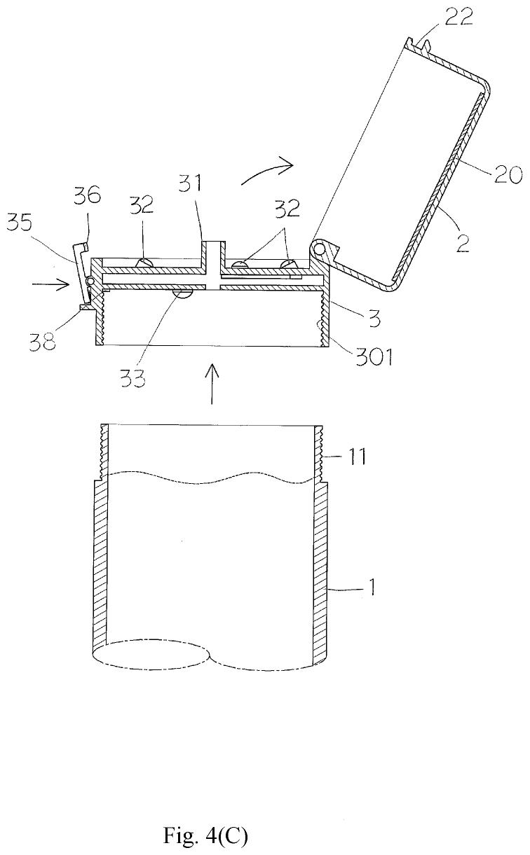

As shown in FIG. 4 (A), according to said use of the container, when the first cover body (2) closes the second cover body (3) and the second cover body (3) is combined with the container (1), a closed sterilizing space is formed between the first cover body (2) and the second cover body (3) and between the second cover body (3) and container (1) respectively, meanwhile all of the first UV light source structures (32) and the second UV light source structures (33) are turned on for a preset period of time for sterilization. Wherein all of the first UV light source structures (32) irradiate the whole suction port (31) for comprehensive timed sterilization of inside and outside. The second UV light source structures (33) will irradiate the liquid loaded in the container (1) for timed sterilization, when the preset sterilization time is finished, the first UV light source structure (32) and the second UV light source structure (33) are turned off immediately.

As shown in FIG. 4 (B), after the catching groove (22) is released by pressing the snap (35), the first cover body (2) can be lifted up, the first sensor (36) detects disengagement, the controller turns off all of the first UV light source structures (32), so as to avoid the UV light irradiating human eyes or other parts by accident to cause injuries. When the first cover body (2) is closed after use, the sensor (36) detects contact, the controller actuates all of the first UV light source structures (36) again, so as to perform all-directional timed sterilization for the suction port (31).

As shown in FIG. 4 (C), when the second cover body (3) is disengaged from the container (1), in the same way, the controller turns off the second UV light source structure (33) after the second sensor (38) is disconnected, so as to avoid the UV light irradiating human body part by accident to cause injuries. When the second cover body (3) is screwed on the container (1) again, the second sensor (38) detects connection again, and the controller actuates the second UV light source structure (33) again, so as to perform timed sterilization for the liquid loaded in the container (1).

Whenever the first cover body (2) is opened or the second cover body (3) is separated from the container (1), the information of disconnection of the first sensor (36) and the second sensor (38) is sent to the circuit structure (30), the controller turns off all of the first UV light source structures (32) and the second UV light source structures (33), so as to avoid the UV light irradiating human body to cause accidental injuries. Therefore, the present invention has favorable safety.

The timer and relay in the circuit structure (30) of the present invention provide the timing function of default sterilization time and the sterilization restarting function. In addition, even if the first cover body (2) is opened or the second cover body (3) is unscrewed before or after the preset sterilization time is finished, all of the first UV light source structures (32) and the second UV light source structures (33) will be turned off immediately to stop lighting, when the first cover body (2) is closed or the second cover body (3) is screwed down again, all of the first UV light source structures (32) and the second UV light source structures (33) are restarted to emit light, entering the timed sterilization process again.

In addition, the Bluetooth module in the circuit structure (30) is provided for pairing unspecific mobile devices (e.g. mobile phone, tablet PC, notebook PC), so as to set up and adjust the time. This function is the sterilization time setting and adjustment of the first UV light source structure (32) and the second UV light source structure (33). In implementation, the mobile device is equipped with a default App (Application), when the mobile device and container are paired, the sterilization time of the first UV light source structure (32) and the second UV light source structure (33) can be set up and adjusted by operating the App. For example, if the first cover body (2) and the second cover body (3) are used for a larger container (1), as the volume of the loaded liquid increases, the sterilization time of the second UV source structure (33) shall be prolonged relatively, this adjustment method is applicable to bottles, jars, cups and barrels of different capacities, so as to implement convenient real-time adjustment.

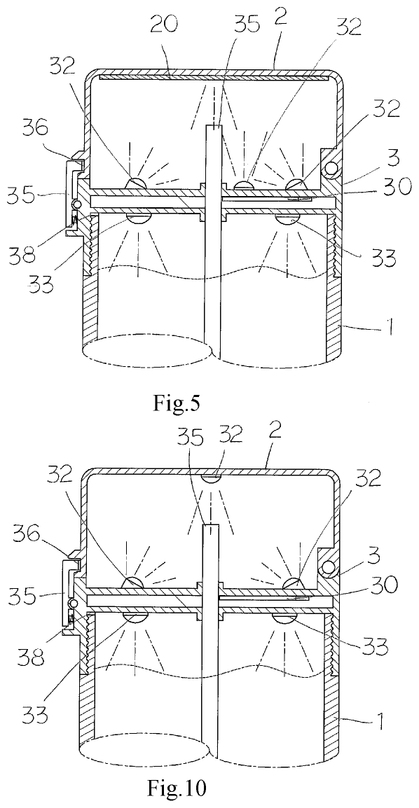

In addition, as shown in FIG. 5, the suction port (31) of the second cover body (3) can be combined with a straw (35), so as to form a friendly and safe type for children. When the container (1) of the present invention is used by a child, if the first cover body (2) is opened or the second cover body (3) is unscrewed by accident, the lights can be turned off immediately under the effect of the first sensor (36) and the second sensor (38), so there will be no illumination harm to the child.

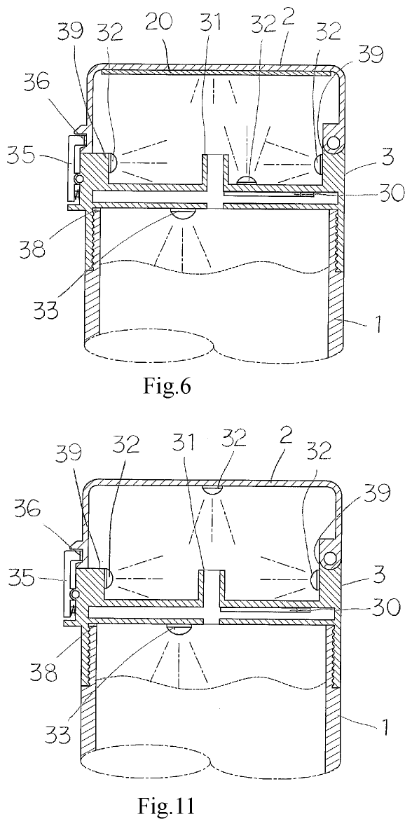

As shown in FIG. 6, the second cover body (3) is provided with more than one first UV light source structure (32) for irradiating the circumference of suction port (31), in practical arrangement, besides a tilt angle, it can be disposed on peripheral surface of the second cover body (3). In implementation, an upright ring surface (39) is formed around the second cover body (3), and more than one first UV light source structure (32) is embedded in the upright ring surface (39), the quantity and location of the first UV light source structures (23) are unrestricted, they can be distributed equidistantly or oppositely, so as to thoroughly irradiate the circumference of the suction port (30) to implement positive sterilizing effect.

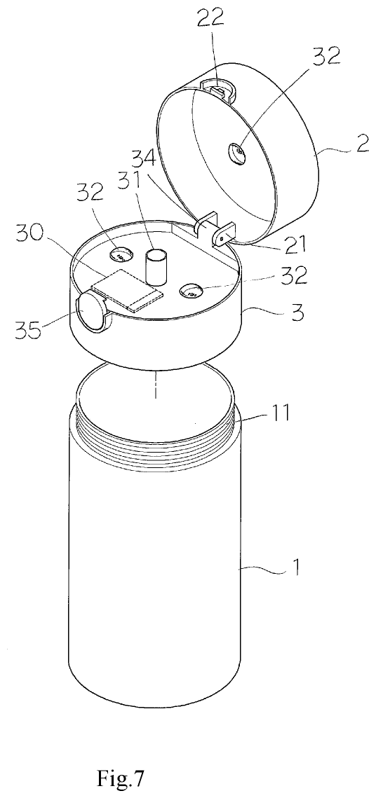

The second preferred embodiment in FIG. 7 shows another schematic diagram of external structure of the container with automatic sterilization function. As shown in FIGS. 8 and 9. The opening of the container (1) is combined with a first cover body (2) and a second cover body (3) which are connected to each other. The inner face of inner chamber of the first cover body (2) is provided with a first UV light source structure (32). A circuit structure (30) is disposed in the main body of the second cover body (3). A suction port (31) is disposed on the surface of the second cover body (3) towards the first cover body (2). At least more than one first UV light source structure (32) irradiating the circumference of the suction port (31) is laid by the suction port (31). More than one second UV light source structure (33) is disposed on another surface of the second cover body towards the container.

The configuration of the first sensor (36) for controlling the on/off of the first UV light source structure (32) and the second sensor (38) for controlling the on/off of the second UV light source structure (33) is the same as the first embodiment. In this embodiment, the first UV light source structure (32) on the inner face of inner chamber of the first cover body (2) is wired through the hinge structure and electrically connected to the circuit structure (30), the rest of the first UV light source structures (32) and second UV light source structure (33) are directly electrically connected to the circuit structure (30). In implementation, the first UV light source structure (32) on the inner face of inner chamber of the first cover body (2) irradiates the end edge and inside of suction port (31) periodically, and more than one first UV light source structure (32) around the suction port (31) irradiates the circumference of the suction port (31) simultaneously, so that the suction port (31) is sterilized thoroughly.

The form of said hinge structure between the first cover body (2) and the second cover body (3) is unrestricted, but it must allow wire to pass through, so as to be electrically connected to the circuit structure (30), and to obtain the required power delivery and function execution.

As shown in FIG. 10, in the second embodiment, the suction port (31) of the second cover body (3) can be combined with a straw (35), becoming a friendly and safe container type for children. Moreover, when the container (1) of the present invention is used by a child, if the first cover body (2) is opened or the second cover body (3) is unscrewed by accident, the lights can be turned off immediately under the effect of the first sensor (36) and the second sensor (38), there will be no illumination harm to the child.

As shown in FIG. 11, in the second embodiment, more than one first UV light source structure (32) is arranged on the circumference of suction port (31) of the second cover body (3), besides a tilt angle in the first embodiment, it can be disposed on the peripheral surface of the second cover body (3). In implementation, an upright ring surface (39) is formed around the second cover body (3), more than one first UV light source structure (32) is embedded in the upright ring surface (39), and the quantity and location of the first UV light source structure (32) are unrestricted, which can be increased and adjusted as required, so as to thoroughly and properly irradiate the circumference of the suction port (31) to obtain more positive sterilizing effect.

As described above, in the present invention, as the suction port is the unique part the user's mouth contacts, the sterilizing effect on the suction port will directly influence the user's physical health status. The all-directional sterilizing effect on the suction port which is contacted repeatedly. Moreover, the secondary sterilization design for the liquid loaded in the container makes drinking safer for the user.

To sum up, in the use of the container with automatic sterilization function of the present invention, a closed sterilizing space is formed between the first cover body and the second cover body and between the second cover body and container respectively, so that all of the first UV light source structures and second UV light source structures perform timed sterilization in this space. It is safer than the known technology which only sterilizes the water in the container. In addition, the container with automatic sterilization function of the present invention turns on the first UV light source structure and the second UV light source structure automatically in a closed space, which are turned off automatically when the space is opened, the probability of UV irradiation to human body can be reduced completely.

Although the invention has been explained in relation to its preferred embodiment, it is to be understood that many other possible modifications and variations can be made without departing from the spirit and scope of the invention as hereinafter claimed.

1. A container with automatic sterilization function, the opening of the container is combined with a first cover body and a second cover body which are connected to each other; the inner face of inner chamber of the first cover body has a reflective layer made of an arbitrary reflecting material; a circuit structure is disposed in the main body of the second cover body; a suction port is disposed on the surface of the second cover body towards the first cover body, at least more than one first UV light source structure irradiating the inner chamber of the first cover body and at least more than one first UV light source structure irradiating the circumference of the suction port are arranged around the suction port, another surface of the second cover body towards the container is provided with more than one second UV light source structure; all of the first UV light source structures and the second UV light source structures are electrically connected to the circuit structure to provide timed sterilizing effect.

2. A container with automatic sterilization function, the opening of the container is combined with a first cover body and a second cover body which are connected to each other; the inner face of inner chamber of the first cover body is provided with a first UV light source structure; a circuit structure is disposed in the main body of the second cover body; a suction port is disposed on the surface of the second cover body towards the first cover body, at least more than one first UV light source structure irradiating the circumference of the suction port is arranged around the suction port; another surface of the second cover body towards the container is provided with more than one second UV light source structure; all of the first UV light source structures and the second UV light source structures are electrically connected to the circuit structure to provide timed sterilizing effect.

3. The container with automatic sterilization function defined in claim 1 or 2, wherein a coupling section and a catching groove opposite to each other are arranged around the first cover body; a fixing component and a snap are arranged oppositely around the second cover body; the fixing component and the coupling section form a hinge; the snap can be combined with the catching groove, so that the first cover body can be opened and closed; the inner face of the second cover body for combining the container has a screw thread section; the opening circumference of the container has a corresponding screw thread section, so that the second cover body can be coupled tightly or separated.

4. The container with automatic sterilization function defined in claim 3, a first sensor is laid in the snap for combining the first cover body with the second cover body, the on/off of the first UV light source structure is subject to the open/close of the first cover body; in the combination of the second cover body and container, the inner face of the second cover body corresponding to the container opening is provided with a screw thread section, and a second sensor is disposed on the end edge of the screw thread section connected to the main body, the on/off of the second UV light source structure is subject to the separation/combination of the second cover body and container.

5. The container with automatic sterilization function defined in claim 1 or 2, wherein the circuit structure at least comprises a controller (chip), a timer, a relay, a charge power supply and a Bluetooth module.

6. The container with automatic sterilization function defined in claim 1 or 2, wherein the circuit structure can be disposed in the inner chamber of the first cover body.

7. The container with automatic sterilization function defined in claim 1 or 2, wherein the timed sterilization time of the first UV light source structure can be set up and adjusted freely, within one minute, and the optimal range is 10˜20 seconds; the timed sterilization time of the second UV light source structure is within three minutes, and the optimal range is 90˜120 seconds.

8. The container with automatic sterilization function defined in claim 7, wherein the container has a mobile device, the mobile device has a built-in default App, so that the timed sterilization time can be set up and adjusted after Bluetooth module pairing.

9. The container with automatic sterilization function defined in claim 1 or 2, wherein the quantity and the orientation and tilt angle of location of the first UV light source structures irradiating the circumference of suction port on the second cover body are unrestricted, so as to thoroughly irradiate the whole circumference of the suction port.

10. The container with automatic sterilization function defined in claim 1 or 2, wherein an upright ring surface is formed around the second cover body, the more than one first UV light source structure irradiating the circumference of suction port is embedded in the upright ring surface.

11. The container with automatic sterilization function defined in claim 1 or 2, wherein the suction port can be provided with a straw.

12. The container with automatic sterilization function defined in claim 1 or 2, wherein the container can be any one of bottle, jar, cup and barrel types of different capacities.
